SEQUENCE LISTING (7 sequences)

SEQ ID NO: 1 - protein, 379 aa, synthetic construct
  REGION 1..379: note = CSFV E2 recombinant protein
MPALLSLVSLLSVLLMGCVAERLSCKEDHRYAISSTNEIGPLGAEGLTTTWKEYNHGLQLDDGTVRAICIAGSFRVTALNVVSRRYLASLHKRALPTSVIFELLFDGTSPAIEEMGDDFGFGLCPFDTTPVVKGKYNTTLLNGSAFYLVCPIGWTGVIECTAVSPTTLRTEVVKTFKREKPFPHRADCVTTIVEKEDLFHCKLGGNWTCVKGNPVTYTGGQVKQCRWCGFDFKEPDGLPHYPIGKCILANETGYRIVDSTDCNRDGVVVSTEGEHECLIGNTTVKVYALDGRLAPMPCRPKEIISSAGPVRKTSCTFNYTKTLRNKYYEPRDSYFQQYMLKGEYQYWFDLDVTGHHADYFAEGLEVLFQGPHHHHHHHH

SEQ ID NO: 2 - DNA, 184 bp, synthetic construct
  misc_feature 1..184: note = PCV2 294-477
  source 1..184: mol_type = other DNA
cacatcgagaaagcgaaaggaactgatcagcagaataaagaatactgcagtaaagaaggcaacttactgatggaatgtggagctcctagatctcaaggacaacggagtgacctgtctactgctgtgagtaccttgttggagagcgggagtctggtgaccgttgcagagcagcaccctgtaacgt

SEQ ID NO: 3 - DNA, 218 bp, synthetic construct
  misc_feature 1..218: note = CpG
  source 1..218: mol_type = other DNA
tcgtcgaagtcgttttggggggtctagttcgtcgaaatcgatttggggggtctagttcgtcgaagtcgttttggggggtctagttcgtcgaaatcgatttggggggtctagttcgtcgaagtcgttttggggggtctagttcgtcgaaatcgatttggggggtctagttcgtcgaagtcgttttggggggtctagttcgtcgaaatcgatttgggggg

SEQ ID NO: 4 - DNA, 1116 bp, synthetic construct
  misc_feature 1..1116: note = CSFV E2
  source 1..1116: mol_type = other DNA
atgaaagtattgagaggacagatcgtgcaaggcataatatggctgttgctggtgactggggcacaagggcggttgtcctgtaaggaagaccacaggtatgcaatatcatcaaccaatgagataggaccgctaggggctgaaggtctcaccactacctggaaagaatacaatcatggtttgcagctggatgacgggactgtcagggctatttgcattgcagggtcctttagagtcacggcacttaatgtggttagcaggaggtacctggcgtcattacataagagggctttgcccacttcagtgatatttgaactcctatttgatgggaccagcccagcaattgaggagatgggagatgactttggatttggactgtgcccttttgacacgactcctgtggtcaaagggaagtacaataccactttattaaacggcagcgcattctatctagtctgcccaatagggtggacgggtgtcatagagtgcacggcagtaagccccacaaccttgagaacagaagtggtgaagaccttcaagagagagaagcctttcccgcacagagcggattgcgtgaccactatagtagaaaaagaagacctgttccactgtaagttggggggcaactggacatgtgttaaaggcaacccggtgacctacacgggggggcaagtaaaacaatgcaggtggtgtggttttgacttcaaggagcctgatgggctcccacactaccccataggcaagtgcatcttagcaaatgagacgggttacaggatagtggactccacagactgcaacagagatggcgttgtcgttagcactgaaggagaacatgagtgcttgattggcaacactactgtcaaagtatacgcattggatggaagactggcccctatgccgtgtagacccaaagaaatcatctctagcgcgggacctgtaaggaaaacttcctgcactttcaattacacaaagactctaagaaacaagtactatgagcccagggacagctatttccagcaatatatgcttaagggcgagtaccaatactggtttgatctggacgtgactggccaccatgcagactacttcgccgaaggtcatcaccatcaccatcactga

SEQ ID NO: 5 - DNA, 1073 bp, synthetic construct
  misc_feature 1..1073: note = PCV2 ORF2
  source 1..1073: mol_type = other DNA
tccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaaccgccgccgccatggcccctgcgctgctctccctcgtgagccttctctccgtgctgctgatgggttgcgtagctgaaacctatcctcggcggagatacagacggcggaggcacagacctagatctcacctgggacagatcctgcggagaaggccttggctggtgcaccccagacacagatatcggtggcggagaaagaacggcatcttcaacacccggctgtcccggaccatcggctacaccgtgaagaaaacaaccgtgcggaccccttcctggaacgtggacatgatgcggttcaacatcaacgactttctgccacctggcggcggaagcaaccctctgacagtgcctttcgagtactacaggatccggaaagtgaaggtcgagttctggccctgctctcccatcacacaaggcgatagaggcgtgggctctaccgccgtgatcctggacgataacttcgtgaccaaggccaacgctctgacctacgatccctacgtgaactactccagccggcacacaattacccagcctttctcctaccactctcggtacttcacccctaagcctgtgctggacagaaccatcgactacttccagcctaacaacaagcggaaccagctgtggctgagactgcagaccaccggcaatgtggatcatgtcggcctgggcaccgccttcgagaactctatctacgaccaggactacaacatccggatcactatgtacgtgcagttccgcgagttcaacctgaaggaccctccactgaaccccaagtgaagggttcgatccctaccggttagtaatgagtttgatatctcgacaatcaacctctggattacaaaatttgtgaaagattgactggtattcttaactatgttgctccttttacgctatgtggatacgct

SEQ ID NO: 6 - DNA, 20 bp, synthetic construct
  misc_feature 1..20: note = PCV2 forward primer
  source 1..20: mol_type = other DNA
acatcgagaaagcgaaagga

SEQ ID NO: 7 - DNA, 20 bp, synthetic construct
  misc_feature 1..20: note = PCV2 reverse primer
  source 1..20: mol_type = other DNA
acgttacagggtgctgctct

(19) United States
(12) Patent Application Publication
(10) Pub. No.: US 2024/0293536 A1
(43) Pub. Date: Sep. 5, 2024

(54) PORCINE BIVALENT SUBUNIT VACCINE COMPOSITION IN A SINGLE DOSE

(71) Applicant: National Pingtung University Of Science And Technology, Pingtung (TW)

(72) Inventors: Hso-Chi Chaung, Pingtung (TW); Wen-Bin Chung, Pingtung (TW); Yen-Li Huang, Pingtung (TW); Chi-Chih Chen, Pingtung (TW); Yu-Chieh Chen, Pingtung (TW)

(73) Assignee: National Pingtung University Of Science And Technology, Pingtung (TW)

(21) Appl. No.: 18/394,532

(22) Filed: Dec. 22, 2023

(30) Foreign Application Priority Data
Dec. 26, 2022 (TW) 111149940

(51) Int. Cl. A61K 39/295; A61K 39/00; A61K 39/12; A61P 37/04

(52) U.S. Cl. CPC A61K 39/295; A61K 39/12; A61P 37/04; A61K 2039/552; A61K 2039/55561; A61K 2039/55566; A61K 2039/70

(57) ABSTRACT

The present invention relates to a porcine bivalent subunit vaccine composition in a single dose. The porcine bivalent subunit vaccine composition includes porcine bivalent antigen, CpG adjuvant and a dual phase adjuvant. The porcine bivalent antigen consists of a classical swine fever virus (CSFV)-E2 recombinant protein and a porcine circovirus type 2 (PCV2)-ORF2 recombinant protein, both of which are produced by a mammalian cell expression system. The porcine bivalent subunit vaccine composition in a single dose can confer effectively immune protection against CSFV and PCV2 via a single vaccination without boost vaccination.

### RELATED APPLICATION

This application claims priority to Taiwan Application Serial Number 111149940, filed Dec. 26, 2022, which is herein incorporated by reference in its entirety.

### REFERENCE TO THE SEQUENCE LISTING

A sequence listing is being submitted herein as xml format with the name “SP-5904-US_SEQ_LIST”, created on Nov. 14, 2023, with a file size of 12,881 bytes.

### BACKGROUND

### Field of Invention

The present invention relates to an animal vaccine composition. More specifically, the present invention to a porcine bivalent subunit vaccine composition in a single dose against classical swine fever virus (CSFV) and porcine circovirus type 2 (PCV2).

### Description of Related Art

Classical swine fever (CSF), also known as classical hog cholera, is a highly contagious disease of swine. Typically, infected pigs develop high fever, anorexia, diarrhea, neurologic signs, erythema over the body, mass death, abortion or stillbirth in pregnant sows, and CSF is a highly contagious and highly pathogenic disease of pigs.

Since CSFV is firstly found in 1903, it is endemic in many parts of worldwide, including Asia, Africa, Europe, Central and South America and so on. Once an endemic breaks out, it often causes massively economic losses to the pig industry and drastically affects the global economy. Therefore, CSF is a disease listed in List A (List A) of the World Organization for Animal Health (founded as OIE) and must be reported to the organization. In East Asia such as Taiwan, swine fever is rampant in Taiwan from Japanese colonial period. The incidence rate of swine fever was as high as 81.3% in 1948 according to literatures. CSFV endemic countries have to use routine vaccination to prevent and control the spread of the pathogen. When used properly, vaccination can be an effective approach to limit transmission of the swine fever. In 2017, 32 of the 181 OIE-member countries are free of CSFV transmission. If the E2 subunit vaccine with better protection can be developed, the subunit vaccine will induce protective neutralizing antibodies and be capable of replacing the conventional live vaccine, thereby leading to become CSFV-free countries.

Classical swine fever virus (CSFV), which is the causative agent of CSF, belongs to the genus Pestivirus of the family Flaviviridae. E1 and E2 proteins are structural proteins on the CSFV envelope, both of which are glycoproteins and play a key role in entry of CSFV into the host cell. Therefore, E2 protein is widely used as an important epitope of antigens for developing CSFV subunit vaccine.

Nowadays, the vaccination is the best strategy of controlling all diseases. The vaccines of controlling the swine fever can be sun-divided into two categories, live (attenuated) vaccines and subunit (dead) vaccines. Live attenuated vaccines are widely used in the CSF endemic areas. As for the current development of CSF vaccine against in East Asia, for example, in Taiwan, the vaccines of tissue culture origin have gradually replaced the conventional rabbitized CSF vaccines. For instance, the marketed vaccine product Bayovac CSF E2™, which is produced by Ta Foong Vaccines & Biotech Co., Ltd. (OEM), includes CSFV-E2 antigen and a water-in-oil adjuvant, and the CSFV-E2 antigen is produced in a baculovirus-infected insect cell. A single dose of the Bayovac CSF E2™ is 2 mL to be injected per animal, and the boost dose is given after a 3-week interval (administered with 60 μg of CSFV-E2 antigen totally).

In addition to inducing protective neutralizing antibodies, the E2 subunit vaccine can meet the DIVA (differentiation of infected from vaccinated animals) requirement. In the case of the vaccine product (e.g., Bayovac CSF E2™), it includes CSFV-E2 antigen produced in a baculovirus-infected insect cell, as well as a water-in-oil adjuvant. A single dose of the vaccine product is 2 mL to be injected per animal, and the boost dose is given after a 3-week interval (administered with 60 μg of CSFV-E2 antigen totally).

The vaccine product can effectively elevate the antibodies in vaccinated animals, and overcome the disadvantages of the conventional live vaccines that are not capable of meeting the DIVA. Such CSFV-E2 antigen can be used in differential diagnostic reagents, for early diagnosis of viral infections in the field, quick elimination of potential CSFV in pig farms, and facilitating the promotion of CSF eradication in Taiwan.

However, a research in 2005 showed that a switch in genotype from the subgroup 3.4 to the subgroup 2.1a of CSFV was observed in Taiwan from the past decades. There is an urgent need to improve the immunoprotection of CSF vaccine. Another common swine pathogen, porcine circovirus type 2 (PCV2), is the primary causative agent of several syndromes collectively known as porcine circovirus-associated disease (PCVD), including porcine dermatitis nephropathy syndrome (PDNS), porcine respiratory disease complex (PRDC), reproductive failure, proliferative necrotizing pneumonia (PNP), and granulomatous enteritis and so on. PCVD is an important viral disease dramatically impacting the global swine industry, and it is a significant problem how to prevent and control the disease.

PCV2 belongs to the genus Circovirus of the family Circoviridae, is the smallest, pathogenic and nonenveloped DNA virus with single-stranded circular genome. Currently, eight genotypes of PCV2 have been identified.

In East Asia, similar cases of PCV2 infection were reported in Taiwan from 1997, in which approximately 70-80% of weak piglets were infected with PCV2, and the prevalence rate of PCV2 of 71% was reported by Wang et al. (The Journal of Veterinary Medical Science, Volume 66, Issue 5, pages 469-475, 2004). To date, no specific treatment is available for PCV2-infected pigs, although most pigs are co-infected. During initial PCVAD (porcine circovirus-associated disease) outbreaks, pigs treated with antibiotics actually suffered a higher mortality rate than those not treated, but it is believed that this was more severe because of the virus transmission by sharing needles than treatments with antibiotics. As such, it is necessary to effectively control PCV2 outbreaks with vaccines.

In present, the PCV2 vaccine available on the market is a PCV inactivated vaccine (the brand name: Ingelvac CircoFLEX® vaccine manufactured by Boehringer Ingelheim, Petersurg, VA; hereinafter abbreviated as Boehringer Ingelheim's vaccine) and a PCV subunit vaccine (the brand name: Porcilis® PCV vaccine manufactured by Intervet Inc/Schering-Plough Animal Health, Kenilworth, NJ; hereinafter abbreviated as Intervet's vaccine). Boehringer Ingelheim's vaccine is a PCV2 ORF2 capsid-based subunit vaccine expressed in inactivated baculovirus, and 3-week-old piglets are vaccinated with a single shot (dose) by intramuscular injection. Intervet's vaccine is also a PCV2 ORF2 capsid-based subunit vaccine. It is designed for vaccination of piglets 3 weeks and older with a single dose by intramuscular injection.

However, PCV2 infection causes immunosuppression in pigs that leads to higher risk of CSFV infection. Some previous researches have proved that PCV2 infection affects the efficacy of the Lapinized Philippines Coronel (LPC).

Accordingly, there is an urgent need to develop a multivalent vaccine, for reducing the possibility of coinfection with CSFV and PCV2 in the field, decreasing leaks in the prevention and control CSFV with current LPC vaccines, lowering the risk of wild CSFV invading the pig farms, and increasing the opportunity of clearing CSFV, thereby solving various problems of the prior arts.

### SUMMARY

Accordingly, an aspect of the invention provides a porcine bivalent subunit vaccine composition in a single dose, which includes a porcine bivalent antigen, CpG adjuvant and a dual phase adjuvant. The porcine bivalent antigen consists of a classical swine fever virus (CSFV)-E2 recombinant protein and a porcine circovirus type 2 (PCV2)-ORF2 recombinant protein, both of which are produced by a mammalian cell expression system. The porcine bivalent subunit vaccine composition in a single dose can confer complete immune protection against CSFV and PCV2 via a single vaccination without boost vaccination.

According to the aforementioned aspect, the invention provides a porcine bivalent subunit vaccine composition in a single dose, which includes a porcine bivalent antigen, CpG adjuvant and a dual phase adjuvant, and a subject is administered by the porcine bivalent subunit vaccine composition with an effective administration time of only once. In an embodiment, the aforementioned bivalent antigen can be consisted of a E2 recombinant protein and a ORF2 recombinant protein, for example, an amino acid sequence of the E2 recombinant protein can be listed as SEQ ID NO:1, the ORF2 recombinant protein can be encoded by a nucleic acid sequence listed as SEQ ID NO:2, and a CpG adjuvant can be consisted of a nucleic acid sequence listed as SEQ ID NO:3.

In the aforementioned embodiment, the E2 recombinant protein can be derived from is derived from classical swine fever virus (CSFV) subgroup 2.1a, the ORF2 recombinant protein is derived from porcine circovirus type 2 (PCV2), for example.

In the aforementioned embodiment, the bivalent antigen can be produced by a mammalian cell expression system, for example.

In the aforementioned embodiment, the E2 recombinant protein can be encoded by a nucleic acid sequence listed as SEQ ID NO:4, for example.

In the aforementioned embodiment, an effective dose of the E2 recombinant protein can be 25 μg/mL to 50 μg/mL, and an effective dose of the ORF2 recombinant protein can be 25 μg/mL to 75 μg/mL, for example.

In the aforementioned embodiment, a weight ratio of the CpG adjuvant and the bivalent antigen can be 1:1 to 1:2, for example.

In the aforementioned embodiment, the dual phase adjuvant can be a water-in-oil-in-water (W/O/W) adjuvant, for example.

In the aforementioned embodiment, an effective dose of the dual phase adjuvant is 50 vol. % based on the porcine bivalent subunit vaccine composition.

According to the aforementioned aspect, the invention also provides a porcine bivalent subunit vaccine composition in a single dose as aforementioned, an effective dose of the E2 recombinant protein is 25 μg/mL to 50 μg/mL and an effective dose of the ORF2 recombinant protein is 25 μg/mL to 75 μg/mL, and the porcine bivalent subunit vaccine composition is formulated for a single-dose administration to a subject in need thereof.

According to the aforementioned aspect, the invention further provides a porcine bivalent subunit vaccine composition in a single dose as aforementioned, and the porcine bivalent subunit vaccine composition is formulated for a single-dose administration to a piglet without infections of CSFV and/or PCV.

With application to the porcine bivalent subunit vaccine composition in a single dose, which can include the soluble porcine bivalent antigen produced by a mammalian cell expression system, in combination with the CpG adjuvant and a dual phase adjuvant, the resulted porcine bivalent subunit vaccine composition can confer effectively immune protection against CSFV and PCV2 via a single vaccination without boost vaccination, thereby substantially reducing the occurrence of CSFV and PCV2 in the field at the same time.

It is to be understood that both the foregoing general description and the following detailed description are by examples, and are intended to provide further explanation of the invention as claimed.

### BRIEF DESCRIPTION OF THE DRAWINGS

The disclosure can be more fully understood by reading the following detailed description of the embodiment, with reference made to the accompanying drawings as follows.

FIGS. 1A to 1D respectively illustrate the gel pictures (FIGS. 1A and 1C) of sodium dodecyl sulfate polyacrylamide gel electrophoresis (SDS-PAGE), as well as gel pictures (FIGS. 1B and 1D) of Western blotting analysis of both CSFV-E2 recombinant protein and PCV2-ORF2 recombinant protein expressed by mammalian cells according to an embodiment of the present invention.

FIG. 2A illustrates the line graph of average colon temperature of pigs after vaccination with CSFV/PCV2 bivalent vaccine according to an embodiment of the present invention.

FIG. 2B illustrates the line graph of average clinical score of pigs after vaccination with CSFV/PCV2 bivalent vaccine according to an embodiment of the present invention.

FIG. 2C illustrates the line graph of average weight gain of pigs after vaccination with CSFV/PCV2 bivalent vaccine according to an embodiment of the present invention.

FIGS. 3A to 3F are line graphs respectively showing anti-CSFV antibody titer after CSFV challenge (FIG. 3A), CSFV antibody titer after PCV2 challenge (FIG. 3B), CSFV neutralizing antibody titer after CSFV challenge (FIG. 3C), CSFV neutralizing antibody titer after PCV2 challenge (FIG. 3D), anti-PCV2 antibody titer after CSFV challenge (FIG. 3E) and anti-PCV2 antibody titer after PCV2 challenge (FIG. 3E) of CSFV/PCV2 bivalent subunit vaccine vaccinated pigs according to an embodiment of the present invention.

FIGS. 4A and 4B are line graphs of CSF virus load (FIG. 4A) in the serum after CSFV challenge and PCV2 virus load (FIG. 4A) in the serum after PCV2 challenge of CSFV/PCV2 bivalent subunit vaccine vaccinated pigs according to an embodiment of the present invention.

### DETAILED DESCRIPTION

If a term defined or used in a reference is inconsistent or opposite as it is defined or used herein, the definition of the term herein, other than that in the reference, is preferably applicable. Moreover, unless otherwise defined in the context, a singular term can include a plural one, and a plural term can also include a singular one.

As aforementioned, the present invention provides a porcine bivalent subunit vaccine composition in a single dose, which includes a soluble bivalent antigen, CpG adjuvant and a dual phase adjuvant, and the soluble bivalent antigen is produced by a mammalian cell expression system.

The terms “recombinant protein”, “recombinant antigen”, “protein”, “peptide” and “polypeptide”, as used interchangeably herein, refer to a polymeric form of amino acids generally linked by peptide bonds or disulfide bonds. The “peptide” can also apply to amino acids in which one or more amino acid residues are naturally occurring amino acids and their polymers, or amino acid polymers having analogs or mimetics of corresponding naturally occurring counterparts. The “peptide” can further include modified amino acid polymer, for example, glycoprotein having carbohydrate residues, or phosphorylated peptide. The peptide, polypeptide and protein can be produced by liquid phase synthesis, solid phase synthesis, or using genetic engineering, recombinant cells, prokaryotic expression systems, eukaryotic expression systems. In an embodiment, the bivalent antigen consists of a classical swine fever virus (CSFV)-E2 recombinant protein and a porcine circovirus type 2 (PCV2)-ORF2 recombinant protein, both of which are produced by a mammalian cell expression system. In some examples, the E2 recombinant protein and ORF2 recombinant protein can be produced as independent recombinant proteins.

The terms “amino acid” and “residue” can be used interchangeably herein. When the amino acid and the residue are used in combination, the amino acid residue refers to a naturally occurring and synthetic amino acid, an amino acid analog, an amino acid mimetic, and a non-naturally occurring amino acid chemically similar to the naturally occurring counterpart.

There is no limitation to the strains of “CSFV” recited herein, which can be CSFV subgroup 2.1a. There is also no limitation to the strains of “PCV” recited herein, which can be PCV2. However, in other embodiments, CSFV and PCV can also be strains other than the aforementioned, depending on the actual requirements.

The “E2 recombinant antigen” herein refers to a full-length E2 recombinant protein listed as the amino acid sequence of SEQ ID NO:1, for example, or encoded by the nucleic acid sequence of SEQ ID NO:4. In the latter one, the amino acid sequence of the E2 recombinant protein is sequentially a signal peptide (for facilitating the secretion of translated E2 protein into the medium), E2 protein with a modified sequence (HS2 strain, about 342 a.a.), protease cleavage site (for facilitating the cleavage of His tag) and His tag (for purifying protein) from the N terminus to the C terminus. The amino acid sequence of SEQ ID NO:1 can be modified to a sequence optimally produced by mammalian cell expression system according to the E2 gene fragment (the corresponding 1201^{st} to 2294^{th} nucleotides of the gene sequence AY526726.1) adopted from the gene sequence (AY526726.1) of CSFV subgroup 2.1a viral strain disclosed by Pan et al. (Archives of Virology, Volume 150, Issue 6, Pages 1101-1119, 2005). In some examples, for easily constructing the recombinant plasmid, two ends of E2 recombinant gene sequence can be optionally added with cleavage sites of restriction enzymes, in which there is no specific limitation to the kinds of cleavage sites of restriction enzymes, for example, cleavage sites of Bam HI and Not I, depending upon the sequence of the being-constructed plasmid. In addition, the C terminus of E2 recombinant protein can be optionally added with protease cleavage site (for facilitating the cleavage of His tag) and His tag (or called as polyhistidine), in which the His tag can include but be not limited to six to ten His residues. Therefore, the resulted E2 recombinant protein can be s soluble protein.

The “ORF2 recombinant protein” herein refers to the one encoded by the nucleic acid sequence listed as SEQ ID NO:2, for example, in which several point mutations are introduced into the nuclear localization signal (NLS) sequence of the wild-type PCV2 ORF2 gene. However, as in practice the wild-type PCV2 ORF2 gene without modification is expressed in the mammalian cell expression system, the expression of the wild-type PCV2 ORF2 protein is found to be insufficient. In the disclosure, the NLS sequence of the wild-type PCV2 ORF2 gene is modified by introducing the point mutations into the 12^{th}-14^{th}, 16^{th}, 18^{th}, 34^{th}-35^{th}, 37^{th} and 39^{th}-41^{st} amino acid residues of the NLS sequence, as well as codon optimization. The aforementioned point mutations can include but be not limited to deletion, substitution or insertion of bases, instead of introducing the stop codon. The post-translational modification of the mammalian cell expression system can offer correct tertiary structure and folding of the ORF2 protein, for maintaining the antigenicity of the original protein and increasing the yield of the recombinant protein. Therefore, the resulted ORF2 recombinant protein is a soluble protein.

In some embodiments, the CSFV-E2 recombinant protein can be produced by conventional or following methods. Firstly, a transformed prokaryotic cell is employed to express a protein, in which the transformed prokaryotic cell can include a first recombinant plasmid containing a recombinant gene with a nucleic acid sequence listed as SEQ ID NO:4, for expressing the CSFV-E2 recombinant protein with an amino acid sequence listed as SEQ ID NO:1. For subsequent purification of the recombinant protein, 3′ end of the aforementioned recombinant gene can optionally added with a nucleic acid sequence encoding a His tag. In some examples, the nucleic acid sequence encoding the His tag can be provided by a commercially available vector, for example, and it is designed to connect to the 3′ end of the aforementioned recombinant gene. The nucleic acid sequence encoding the His tag is well known and commonly used in this art rather than being described in detail.

Next, the first recombinant plasmid of the above transformed prokaryotic cell is extracted, the E2 recombinant gene is cleaved by restriction enzyme and cloned into a second recombinant plasmid that is available for a mammalian cell expression system. The second recombinant plasmid is transfected into a mammalian cell, for expressing and secreting the E2 recombinant protein into a cell medium in mass. And then, a soluble E2 recombinant protein is recovered and purified.

In the aforementioned embodiment, after recovering the E2 recombinant protein, the E2 recombinant protein is optionally subjected to column purification step, so as to obtain the purified CSFV-E2 recombinant protein.

Generally, the yield of the CSFV-E2 recombinant protein can reach at least 300 mg to 600 mg, or 400 mg to 500 mg, per liter of the cell medium.

In some embodiments, the PCV2-ORF2 recombinant protein can be produced by the same method for producing the CSFV-E2 recombinant protein; however, there is a difference in that the PCV2-ORF2 recombinant protein is purified from the recovery of the transfected mammalian cells, after expression of the PCV2-ORF2 recombinant protein in the mammalian cells in mass. The yield of the PCV2-ORF2 recombinant protein can reach at least 60 mg to 100 mg, or 70 mg to 90 mg, per liter of the cell medium. Accordingly, the yield of the bivalent antigen can be substantially increased.

The soluble bivalent antigen, which consists of the soluble CSFV-E2 recombinant protein and the soluble PCV2-ORF2 recombinant protein, can be combined with CpG adjuvant and the dual phase adjuvant, for making a porcine bivalent subunit vaccine composition in a single dose, which can confer effectively immune protection via a single vaccination. The “single vaccination (or single-shot vaccination)” herein refers to once of an effective administration time when the porcine bivalent subunit vaccine composition in a single dose is administered to a subject, without boost vaccination or side effect, for simplifying the vaccination process.

In some embodiments, the porcine bivalent subunit vaccine composition in a single dose can include 2 mL per dose, 25 μg/mL to 50 μg/mL of an effective dose of the CSFV-E2 recombinant protein per dose, for example; and 25 μg/mL to 75 μg/mL of an effective dose of the PCV-ORF2 recombinant protein per dose, for example. In some certain examples, the porcine bivalent subunit vaccine composition in a single dose can include 25 μg/mL of the effective dose of the CSFV-E2 recombinant protein and 50 μg/mL of an effective dose of the PCV-ORF2 recombinant protein per dose, for example.

In another embodiment, there is no specific limitation to the sequence of the CpG adjuvant, for example, the sequence can be exemplified by SEQ ID NO:3. The sequence and the method of CpG adjuvant refer to U.S. Pat. No. 10,117,929 B1, which is herein incorporated by reference in its entirety. Briefly, transformed cells of E. coli having the sequence of the CpG adjuvant is cultured in mass production, and DNA extraction and measurement are carried out using the conventional method. In some examples, the porcine bivalent subunit vaccine composition in a single dose can include 50 μg/mL or 100 μg/2 mL of an effective dose of the CpG adjuvant.

In the aforementioned embodiment, a weight ratio of the CpG adjuvant and the bivalent antigen protein can be 1:1 to 1:2, for effectively achieving the single-shot vaccination. If the weight ratio of the CpG adjuvant and the bivalent antigen protein was less than 1:1 or more than 1:1, the single-shot vaccination would unlikely provide effective immunoprotection.

In other embodiments, there is no specific limitation to the dual phase adjuvant, for example, a water-in-oil-in-water (W/O/W) adjuvant. In the aforementioned embodiment, the porcine bivalent subunit vaccine composition in a single dose can include 50 volume % of an effective dose of the dual phase adjuvant.

Thereinafter, it will be understood that particular recombinant protein sequences, specific formulations, specific doses, specific detecting methods, aspects, examples and embodiments described hereinafter are shown by way of illustration and not as limitations of the invention. The principal features of this invention can be employed in various embodiments without departing from the scope of the invention. Thus, one skilled in the art can easily ascertain the essential characteristics of the present invention and, without departing from the spirit and scope thereof, can make various changes and modifications of the invention to adapt it to various usages and conditions.

### Example: Preparation of CSFV-E2 Recombinant Protein and PCV2-ORF2 Recombinant Protein

### 1.1 Construction of CSFV-E2 Recombinant Gene

The CSFV-E2 recombinant protein had the amino acid sequence listed as SEQ ID NO:1, which was encode by a nucleic acid sequence from the E2 gene fragment (corresponding to the 1021^{st} to 2294^{th} nucleotides of the gene sequence of GenBank accession AY526726.1) of CSFV subgroup 2.1a that was disclosed by Pan et al. (2005) and modified for proper expression in a mammalian expression system. There were cleavage sites of restriction enzymes Bam HI and Not I designed at two ends of the nucleic acid sequence, respectively, 5′ end of the nucleic acid sequence was added with a gene sequence of protein-tyrosine phosphatase (PTP, approximately 63 b.p. for facilitating the secretion of translated E2 protein into the medium), and its 3′ end was added with the cleavage site sequence (approximately 8 a.a.) of human rhinovirus3C protease (HRV 3C protease) and His tag (approximately 8 a.a.). This nucleic acid sequence was synthesized by Genomics BioSci & Tech Co., Ltd., named as PTP-E2 and listed as SEQ ID NO:4.

### 1.2 Construction of PCV2-ORF2 Recombinant Gene

The PCV2-ORF2 recombinant protein was encoded by the nucleic acid sequence listed as SEQ ID NO:2 from the ORF2 gene fragment (corresponding to the 1030^{th} to 1734^{th} nucleotides of the gene sequence of GenBank accession No.: MN510433.1) of PCV2d that was isolated in 2019 and modified for proper expression in a mammalian expression system. This nucleic acid sequence was synthesized by Genomics BioSci & Tech Co., Ltd., named as PTP-PCV2d and listed as SEQ ID NO:4.

### 2. Expression and Purification of E2 and ORF2 Recombinant Proteins

In this Example, commercially available mammalian cell expression system could be employed to produce E2 and ORF2 recombinant proteins.

2.1 Expression and Purification of E2 recombinant protein

CHO cells were cultured at cell density of 6×10⁶ cells/mL in 37° C. and 8% CO₂ prior to transfection. Next, the CHO cells were transfected with 1 μg/μL of PTP-E2/pcDNA3.4 plasmid DNA according to the indications of commercially available transfection kit (ExpiFectamine™ CHO Transfection Kit, Product No. A29129, manufactured by Thermo Fisher Scientific Inc. Carlsbad, CA, USA, and imported by KIM FOREST ENTERPRISE co., ltd). At the 14^{th} day post-transfection, the supernatant (i.e., the cell medium) were isolated by centrifugation at 2000 g of the rotation speed for 15 minutes and the cells were removed. Later, the supernatant was added with ammonium sulfate (Sigma-Aldrich, Product No. A4915) for precipitation, thereby obtaining 20-40% of precipitant. And then, the precipitant was dissolved in 100 ml of buffer A [containing 50 mM Tris and 500 mM NaCl, pH 7.5]. Subsequently, the dissolving substance (solute) was filtered through the filter membrane having 0.45 μm pore size and subjected to column purification, in which the cell medium was injected into Ni-NTA column, and the E2 recombinant protein was eluted out by an elution solution (containing 50 mM Tris, 500 mM NaCl and 250 mM imidazole), resulting in E2 recombinant protein with high purity.

### 2.2 Expression and Purification of ORF2 Recombinant Protein

CHO cells were cultured at cell density of 6×10⁶ cells/mL in 37° C. and 8% CO₂ prior to transfection. Next, the CHO cells were transfected with 1 μg/μL of PTP-PCV2d/pcDNA3.4 plasmid DNA according to the indications of commercially available transfection kit (ExpiFectamine™ CHO Transfection Kit, Product No. A29129, manufactured by Thermo Fisher Scientific Inc. Carlsbad, CA, USA and imported by KIM FOREST ENTERPRISE co., ltd). At the 14th day post-transfection, the cells were isolated by centrifugation at 2000 g of the rotation speed for 15 minutes and the supernatant (i.e., the cell medium) was removed. Later, the supernatant was added with ammonium sulfate (Sigma-Aldrich, Product No. A4915) for precipitation, thereby obtaining 20-40% of precipitant. And then, the precipitant was dissolved in 100 ml of buffer A [containing 50 mM Tris and 500 mM NaCl, pH 7.5]. Next, the cells were subjected to three cycles of freezing/thawing at −80° C. and 37° C. Following, the dissolving substance (solute) was ultrasonicated in 10 cycles (10 seconds ON and 30 seconds OFF for each cycle) using a commercial equipment (ultrasonic cell disruptor, Sunway Scientific Co.). Subsequently, the dissolving substance (solute) was centrifuged at 12000 g of rotation speed for 20 minutes (Medium Size Refrigerated Centrifuges, Thermo), and the supernatant was filtered through the filter membrane having 0.45 μm pore size (cellulose acetate filter, Cat. No. 11106-47-N, Satorius) and subjected to column purification, in which the cell medium was injected into Ni-NTA column, and the ORF2 recombinant protein was eluted out by an elution solution (containing 50 mM Tris, 500 mM NaCl and 250 mM imidazole), resulting in ORF2 recombinant protein with high purity.

### 2.3 Validation of Recombinant Protein Expression

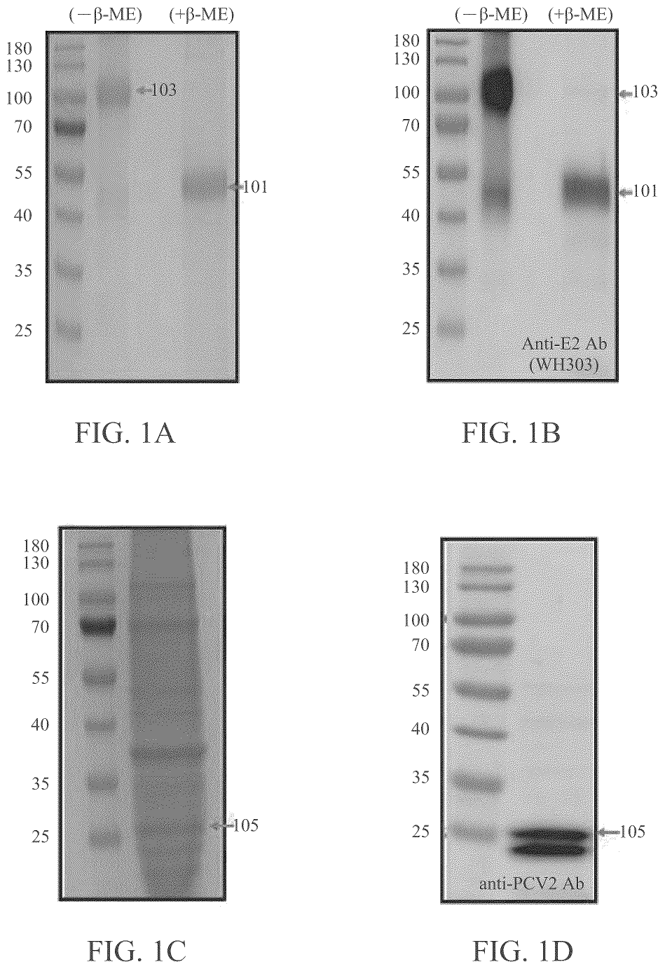

Recombinant proteins obtained in Sections 2.1 and 2.2 were analyzed by sodium dodecyl sulfate polyacrylamide gel electrophoresis (SDS-PAGE), protein transfer and western blot, and the results in FIGS. 1A, 1B, 1C and 1D revealed SDS-PAGE (FIG. 1A) and western blot (FIG. 1B, detected by anti-E2 antibody (WH303)) of the E2 recombinant protein, as well as SDS-PAGE (FIG. 1C) and western blot (FIG. 1D, detected by anti-PCV2 ORF2 antibody) of the ORF2 recombinant protein.

Reference was made to FIGS. 1A to 1D, which revealed gel images of SDS-PAGE (FIGS. 1A and 1C) and stained images (FIGS. 1B and 1D) of Western blot of CSFV-E2 and PCV2-ORF2 recombinant proteins expressed by mammalian cells according to an embodiment of the present invention.

As shown in the results of FIGS. 1A and 1C, the CSFV-E2 and PCV2-ORF2 recombinant proteins could be successfully produced in the supernatant, as indicated by arrows 101 (E2 monomer, 46 kDa), 103 (E2 dimer, 92 kDa) and 105 (ORF2 monomer) of FIGS. 1A and 1C. The results of FIGS. 1B and 1D revealed that the recombinant proteins expressed by the mammalian cells confirmed to the E2 recombinant protein (having the amino acid sequence as listed in SEQ ID NO:1) and ORF2 recombinant protein (having the amino acid sequence encoded by the nucleic acid sequence of SEQ ID NO:2).

In addition, the concentrations of the purified recombinant proteins could be measured by a commercial protein detection kit (Pierce™ BCA Protein Assay, Cat. No. 23225, manufactured by Thermo Scientific Inc. Rockford, IL, USA and imported by Level Biotechnology Inc., Taiwan). The concentration of the E2 recombinant protein was 450 mg/L of cell medium, and the concentration of the ORF2 recombinant protein was 80 mg/L of cell medium.

### 3. Preparation of Adjuvants

### 3.1 Preparation of CpG Adjuvant

The sequence (as listed in SEQ ID NO:3) and the making method of CpG adjuvant were referred to U.S. Pat. No. 10,117,929B1, which was herein incorporated by reference in its entity. Briefly, after the transformants of E. coli containing the sequence of CpG adjuvant were produced in mass, conventional approaches to DNA extraction and measurement of DNA concentration were performed. In practice, the dose of the CpG adjuvant in the vaccine composition was typically 100 μg/2 mL.

### 3.2 Dual Phase Adjuvant

In this Example, Montanide™ ISA 206 (water-in oil-water emulsions W/O/W) manufactured by SEPPIC Inc. (France) was used as the dual phase adjuvant. In practice, the dose of the dual phase adjuvant in the vaccine composition was typically 50 vol. % according to the manufacturer's instructions.

### 4. Formulation and Dose of CSFV/PCV2 Bivalent Vaccine Composition

Group A: administration with the CSFV/PCV2 bivalent subunit vaccine (containing 50 μg/dose of the E2 recombinant protein, 100 μg/dose of the ORF2 recombinant protein, ISA206 adjuvant and CpG adjuvant) in a single vaccination (shot).

Group B: administration with normal saline (0.9% NaCl solution, commercially available) as non-vaccinated group or CSFV positive control.

Group C: administration with the CSFV/PCV2 bivalent subunit vaccine (containing 50 μg/dose of the E2 recombinant protein, 100 μg/dose of the ORF2 recombinant protein, ISA206 adjuvant and CpG adjuvant) in a single vaccination (shot).

Group D: administration with normal saline (0.9% NaCl solution, commercially available) as non-vaccinated group or PCV2 positive control.

The CSFV/PCV2 bivalent subunit vaccine included 50 μg/dose of the E2 recombinant protein, 100 μg/dose of the ORF2 recombinant protein, 100 μg of CpG adjuvant and 50 vol. %/dose of ISA206 adjuvant. During the experimentation period of Section 5, six doses were prepared, a total dose included CSF-E2 recombinant protein (240 μL, 1.25 μg/μL), PCV2-ORF2 recombinant protein (5,310 μL, 0.113 μg/μL), PBS (408 μL), CpG adjuvant (42 μL, 14.29 μg/μL) and ISA206 adjuvant (6,000 μL).

### 5. Validation and Application of Bivalent Vaccine Composition

### 5.1 Safety Test

In this Example, twenty primary specific pathogen free (SPF) piglet with four weeks old were bred in the animal room on the Animal Technology Research Center (ATRC, Miaoli, Taiwan). All piglets were born by Caesarean section, fed without colostrum, tested negatively for anti-CSFV and anti-PCV2 antibodies, and randomly divided into four groups (A, B, C and D) of five subjects in each group according to their body weight and gender. The formulation of vaccine compositions applied to Groups A, B, C and D was described as Section 4. The health status of the piglets was monitored, and the following treatments were subjected to the piglets under their stable conditions.

The tests of safety and efficacy for groups were designed in TABLE 1 as below. In Groups A and B, the piglets were challenged with CSFV for efficacy test after the safety test, 2 mL per dose for each vaccination. In Groups C and D, the piglets were challenged with PCV2 for efficacy test after the safety test.

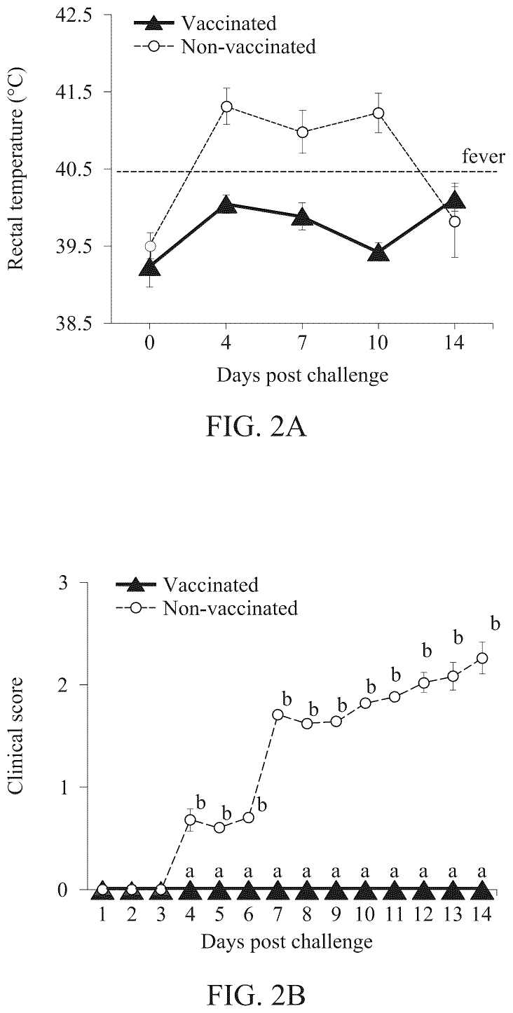

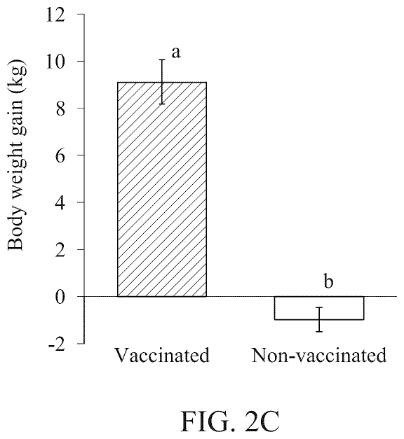

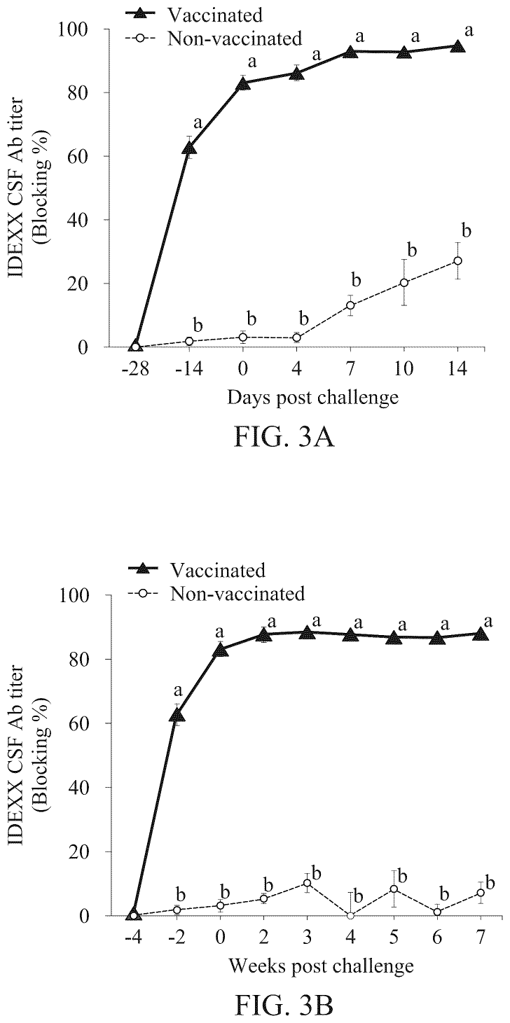

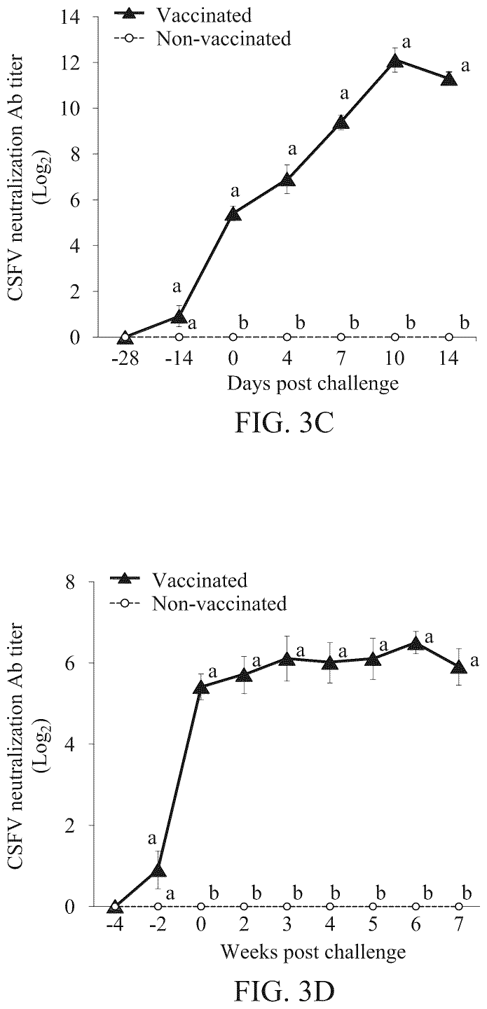

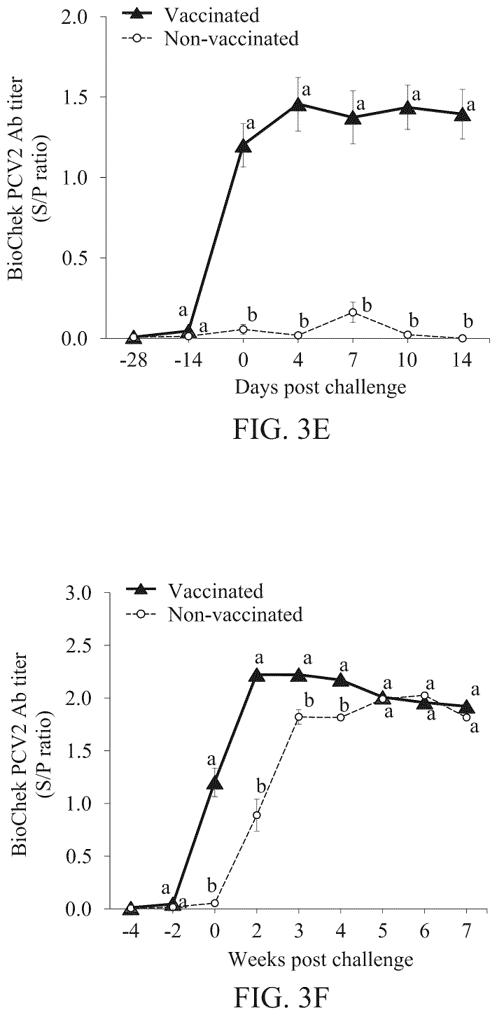

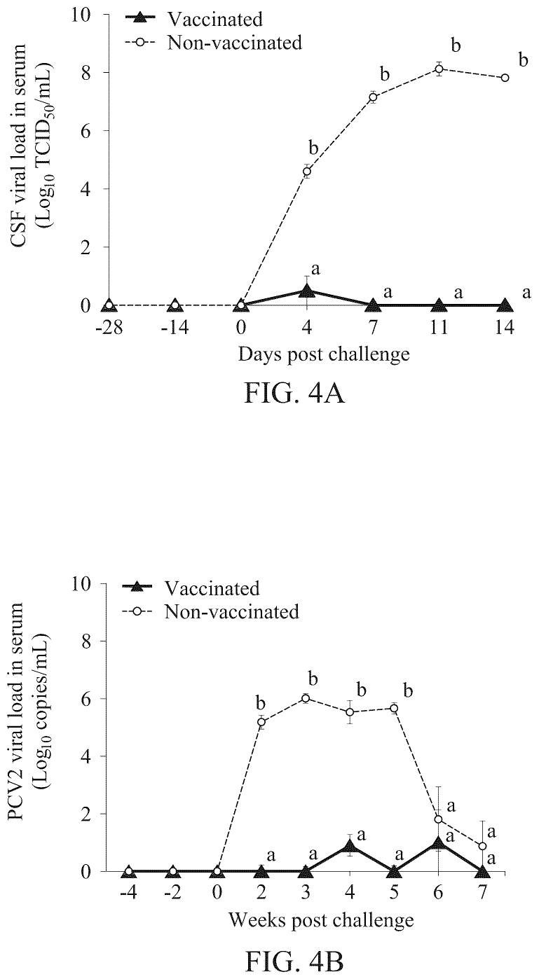

The piglets were administered at the left side of their neck with the vaccine or normal saline, and the clinical symptoms were observed and recorded daily. Their body temperature (colon temperature) was detected before every vaccination or 12, 24, 48 hours and at four weeks after vaccination, and the injection site was inspected; their body weight were measured before vaccination and at four weeks after vaccination (as shown in FIG. 2C); their blood was collected before vaccination or at two and four weeks after vaccination (as shown in FIGS. 3A to 4B). All piglets were monitored during the total 28 days of the administration with vaccines, and challenged with virulent strains under health condition and no fever (colon temperature below 40.5° C.).

TABLE 1

| | | Treatment* | |
|---|---|---|---|
| | Pig | | Challenge with |
| Group | population | Vaccination with vaccines | viruses |
| A | 5 | Vaccination with CSFV/PCV2 bivalent | CSFV |
| | | vaccine** | |
| B | 5 | Administration with normal saline | CSFV |
| C | 5 | Vaccination with CSFV/PCV2 bivalent | PCV2 |
| | | vaccine | |
| D | 5 | Administration with normal saline | PCV2 |

The symbol * in TABLE 1 represented piglets that were grown in the positive pressure rooms for animals in ATRC and vaccinated with vaccines. After 4 weeks after vaccination, the piglets of Groups A and B were transported to the negative pressure rooms for animals in Animal Health and Research Institute (AHRI, Danshui, Taiwan) and challenged with CSFV; and the piglets of Groups C and S were transported to the negative pressure rooms for animals in National Pingtung University of Science and Technology and challenged with PCV2.

The symbol ** in TABLE 1 represented piglets that were vaccinated with CSFV/PCV2 bivalent vaccine containing 50 μg of the E2 recombinant protein and 100 μg of the ORF2 recombinant protein for each dose.

In addition, the healthy status of the piglets was accessed according to clinical evaluation in TABLE 2. The average of the total scores was taken as a clinical index, where zero (0) indicated the minimum of the average of the scores and three (3) was the maximum, resulting in FIG. 2B.

TABLE 2

| No. | Characteristics | Standard (Criteria) | Score |
|---|---|---|---|
| 1 | actions | being curiously and standing up quickly | 0 |
| | | standing up by itself slowly | 1 |
| | | being tired, standing up with force and then lying | 2 |
| | | down | |
| | | unable to move and get up | 3 |
| 2 | body | relax, straight back | 0 |
| | tension | being normal except for being stiff and arched back | 1 |
| | | when standing up | |
| | | arched back, stiff walking | 2 |
| | | spasms | 3 |
| 3 | Body shape | full stomach, round and fat | 0 |
| | | empty stomach | 1 |
| | | empty stomach, thin and weak | 2 |
| | | skinny, visible spine and ribs, the head relatively | 3 |
| | | larger than the body | |
| 4 | Respiration rate | 10-15 breaths per minutes | 0 |
| | (close to pre- | more than 20 breaths per minutes | 1 |
| | measurement of | more than 20 breaths per minutes, chest rising and | 2 |
| | pigs) | falling obviously when breathing | |
| | | more than 30 breaths per minutes, mouth breathing | 3 |
| 5 | walking | coordinating well | 0 |
| | | limping | 1 |
| | | ataxia, hind limb lameness but being able to walk | 2 |
| | | unable to walk | 3 |
| 6 | skin | light pink skin with flat hair | 0 |
| | (especially ears, | reddened area | 1 |
| | nose, legs | purple and cold skin with a little ecchymosis | 2 |
| | and tail) | black and red skin, loss of sensation, hemorrhage in | 3 |
| | | large areas | |
| 7 | eye/cornea | light pink eye | 0 |
| | | eye with redness and clear discharge | 1 |
| | | eye with inflammation and turbid discharge | 2 |
| | | eye with inflammation, purulent discharge, | 3 |
| | | prominent blood vessels | |
| 8 | appetite | good appetite | 0 |
| | | eating slowly during feeding | 1 |
| | | tasting but not eating food during feeding | 2 |
| | | refusing to eat, no interest in food | 3 |
| 9 | defecation | soft stool with normal volume | 0 |
| | | drier stool with decreased volume | 1 |
| | | fewer, dry or fiber-coated stool or diarrhea | 2 |
| | | no stools, watery or bloody stools | 3 |
| 10 | residual feed | completely empty feed trough | 0 |
| | intake | empty feed trough in most time | 1 |
| | | partial feed intake | 2 |
| | | no feed intake | 3 |

### 5.2 Efficacy Test

Ten pigs of Groups A and B in Section 5.1 were challenged with CSFV in negative pressure animal room of Animal Health Research Institute (AHRI), Council of Agriculture, Executive Yuan, Taiwan; another ten pigs of Groups C and D were challenged with PCV2 in negative pressure animal room of Pingtung University of Science and Technology, Taiwan.

### 5.3 Viral Challenge Test

Each pig of Groups A and B was inoculated at right neck side with 2 mL of CSFV fluid (ALD strain) in the dose of 10^{5.41} FAID₅₀ (50% fluorescent antibody infectious dose). The blood samples were collected and body temperatures were measured respectively at the fourth, seventh, tenth and fourteenth day after CSFV challenge; and the body weights were measured before sacrifice. At the fourteenth day after CSFV challenge, all animals were sacrificed, pictured, recorded and sampled.

Each pig of Groups C and D was inoculated at right neck side with 2 mL of PCV2 fluid in the dose of 10⁶ TCID₅₀/mL (50% fluorescent antibody infectious dose) and inoculated intranasally with 2 mL of PCV2 fluid in the dose of 10⁶ TCID₅₀/mL for three contiguous days. The body temperature of pigs was measured before the virus challenge. The blood samples were collected and body temperatures were measured respectively at the second, third, fourth, fifth, sixth and seventh week after PCV2 challenge; and the body weights were measured before sacrifice. At the seventh week after CSFV challenge, all animals were dissected, pictured, recorded and sampled.

All serum samples were subjected to the analyses of viral nucleic acids and antibody titers. All organ samples were subjected to the analysis of viral nucleic acid titers.

### 6. Detection Methods

### 6.1 Serological Detection of CSFV and PCV2

Serological ELISA and the method of detecting neutralizing antibody were performed according to “the method of detecting swine fever” announced by the Council of Agriculture, Executive Yuan of R.O.C.

After a pretreatment, the specimen was subjected to an ELISA with a commercially available kit (IDEXX CSFV antibody test kit, IDEXX Laboratories, Inc., USA) according to the manufacturer's instruction. And then, the results were detected by a commercially available equipment (SPECTRO starnano, BMG LABTECH, Germany) and analyzed by the criteria of the aforementioned kit. A positive result was defined by a blocking percentage (blocking %) of the antibody titer greater than or equal to 40, a negative result was defined by the blocking % of the antibody titer less than or equal to 30, and a further detection was needed when the blocking % of the antibody titer greater than 30 and less than 40.

Besides, after the pretreatment, the collected specimen was subjected to an ELISA with a commercially available kit [PCV2 antibody test kit (ELISA), BioChek, Netherlands] according to the manufacturer's instruction. And then, the results were detected by a commercially available equipment (SPECTRO starnano, BMG LABTECH, Germany) and analyzed by the criteria of the aforementioned kit. A positive result was defined by a ratio of sample-to-positive ratio (S/P ratio) greater than or equal to 0.500, and a negative result was defined by the ratio of S/P ratio less than 0.49.

### 6.2 Detection of Nucleic Acids of CSFV and PCV2

The method of detection of nucleic acids of CSFV and PCV2 was also performed according to the “the method of detecting swine fever” of the Section 6.1.

The extraction and quantification of CSFV nucleic acid could be performed according to the conventional methods. Firstly, CSFV nucleic acid was extracted from the specimen (serum or tissue homogenates) using commercially available nucleic acid extraction kit (for example, MagNA Pure 24 Total NA Isolation Kit, Roche Molecular Systems, Inc., USA), followed by quantification of CSFV virus using commercially available real-time reverse transcriptase PCR Kit (for example, QuantiTect Probe RT-PCR Kit, Qiagen, Germany) according to the method suggested by manufacture. In brief, the nucleic acid extracted from the CSFV-infected blood had the known concentration (TCID₅₀/mL), and it was subjected to serial dilutions in the concentrations of 10¹ to 10⁸ TCID₅₀/mL as quantitative standards for establishing a standard curve and the quantitation result was shown as Log₁₀ TCID₅₀/mL.

The extraction and quantification of PCV2 nucleic acid could be performed according to the conventional methods.

The viral nucleic acid was extracted from 200 μL of serum or tissue homogenates using commercially available DNA extraction kit (for example, AxyPrep Body Fluid Viral DNA/RNA Miniprep Kit, Axygen®; Corning, USA), and it was subjected to real-time reverse transcriptase PCR (real-time RT-PCR) for quantitating viruses.

The real-time RT-PCR for detecting PCVS viral nucleic acid included the steps as follows. Quantitative reaction was carried out in aliquots (2 μL) of DNA sample that included the PCV2 recombinant plasmid as quantitative standard and a negative sample as a negative control. The quantitative reaction performed as follows: 0.5 UM forward/reverse primers (forward primer: 5′-ACATCGAGAAAGCGA AAGGA-3′, as shown in SEQ ID NO:6; reverse primer: 5′-ACGTTACAGGGTGCTGCTCT-3′, as shown in SEQ ID NO:7) and 2× conc. Master Mix (PowerUp SYBR Green Master Mix, Applied Biosystems™ Thermo Fisher Scientific Inc., USA), using QuantStudio™ 3 Real-Time PCR Systems (Applied Biosystems™, Thermo Fisher Scientific Inc., USA) in the condition of 50° C. 2 minutes, 95° C. 2 minutes (1 cycle); 95° C. 15 seconds; 60° C. 15 seconds, 72° C. 1 minute (totally 40 cycles). Subsequently, the melt curve stage was performed in the condition of 95° C. 15 second, 60° C. 1 minute, 95° C. 15 seconds (1 cycle). After the PCR reaction was finished, the viral titer could be analyzed by and commercially available software (for example, QuantStudio™ Design and Analysis desktop Software, ver.v1.5.1) and estimated from a standard curve.

### 6.3 Statistical Analysis

In the aforementioned example, the generalized linear models (GLM) of the commercially available statistical software SAS (ver. 9.4) were used to detect the mean values of the samples being different or not among them, and a statistically significant difference was determined as P value of less than (<) 0.05.

### 7. Determination of Vaccine Effectiveness

The effectiveness of CSFV E2 subunit vaccine was accessed according to Article 182-8 of Section 84^{th}, Examination Standard for Animal Drugs of Swine Fever E2 subunit vaccine in Taiwan. In brief, after the vaccinated pigs were observed for 28 days, they were injected intramuscularly with a highly virulent CSFV ALD strain in 5×10^{5.0} FAID50 to 8×10^{5.0} FAID₅₀ for observation of 14 days, and they were healthy without adverse effect or with mild effect. The pigs of the control group were injected intramuscularly with a highly virulent CSFV ALD strain, and they were found in typically acute swine fever and commonly died in 14 days.

The effectiveness of PCV2 ORF2 subunit vaccine was accessed according the following procedures. The vaccinated pigs were challenged with virus and observed for 7 weeks, the blood was collected in every week except for the first week. The concentration of PCV2 nucleic acid in the sera of the vaccinated pigs (Group C) should be determined significantly less than the non-vaccinated pigs (Group D) in at least three times as P value of less than (<) 0.05.

### 8. Validation of Bivalent Subunit Vaccines

### 8.1 Pigs

When introducing pigs, averaged body weights of vaccinated (administered with P-2V1CSFV/PCV2 bivalent subunit vaccine; groups A and C) and non-vaccinated (administered with normal saline; groups B and D) pigs were 3.6±0.1 kg and 3.5±0.1 kg, respectively. In order to prevent nutritional diarrhea in piglets, all pigs ate freely after pigs were introduced. Gradually, all pigs were provided with sufficient feed in two daily meals of the morning and evening. All pigs were subjected to the experiment in good health.

Biosafety Test of Vaccination of Pigs with Bivalent Subunit Vaccines

### (1) Change in Body Temperature

The changes in body temperature of pigs were determined by average rectal temperature. Reference was made to FIG. 2A, which was a line graph showing average rectal temperature of pigs after vaccination with CSFV/PCV2 bivalent subunit vaccine according to an embodiment of the present invention.

As shown in FIG. 2A, the average rectal temperatures (C) of non-vaccinated (groups B and D) and vaccinated (groups A and C) pigs before administration with vaccines or normal saline were 40.1±0.21 and 40.0±0.2, respectively. At 12, 24 and 48 hours after administration of vaccines, the average rectal temperatures of vaccinated (groups A and C) pigs before administration with vaccines kept at a normal rages below 40.5° C. (C) without significant increase. In contrast, non-vaccinated (groups B and D) pigs experienced fever syndrome.

### (2) Clinical Syndromes

Reference was made to FIG. 2B, which was a line graph showing average clinical score of pigs after vaccination with CSFV/PCV2 bivalent subunit vaccine according to an embodiment of the present invention. In FIG. 2B, normal pigs scored as clinical score 0 (zero); pigs with mild syndromes scored as clinical score 1; pigs with moderate syndromes scored as clinical score 2; pigs with severe syndromes scored as clinical score 3. For all data marked with different letters (e.g., a and b), the difference of the means between the two groups was statistically significant; however, for all data marked with the same letter (e.g., a and a), the difference of the means between the two groups was not statistically significant.

As shown in FIG. 2B, all four groups including vaccinated (groups A and C) and non-vaccinated (groups B and D) pigs had no significant clinical syndromes before the challenge with viruses.

### (3) Weight Gain

Average body weight (kg) of vaccinated (groups A and C) and non-vaccinated (groups B and D) pigs at the day of administration with vaccines or normal saline were 4.3±0.2 and 4.2±0.11, respectively, and there was no statistically significant difference between the vaccinated and non-vaccinated pigs (P>0.05). Average weight gain (kg) of vaccinated and non-vaccinated pigs from vaccination to virus challenge were 6.9±0.2 and 7.1±0.2, respectively, and the difference of the means between the two groups was not statistically significant (P>0.05).

8.3 Efficacy Test of Pigs Vaccinated with CSFV/PCV2 Bivalent Subunit Vaccine and Challenged with CSFV

### (1) Change in Body Temperature

Reference was made to FIG. 2A again. As shown in FIG. 2A, the average rectal temperatures (° C.) of all vaccinated (group A) pigs with the bivalent subunit vaccine were below 40.5° C., after CSFV challenge and before scarification, and they had normal activities without clinical syndromes. The average rectal temperatures (° C.) of all non-vaccinated (group B) pigs increased up to 41.3±0.2° C., 41.0±0.3° C. and 41.2±0.3° C. at 4, 7 and 10 days after CSFV challenge, respectively; at 14 days after CSFV challenge (i.e., the day of sacrification), the average rectal temperatures (C) of pigs because the pigs were gradually cachexia reduced to 39.8±0.5° C. In addition, clinical syndromes of non-vaccinated (group B) pigs occurred on the 4^{th} days after CSFV challenge, their condition continuously deteriorated. On the 7^{th} day, non-vaccinated (group B) pigs showed obvious depression, fear of cold and unwilling to move, and some pigs had neurological symptoms.

### (2) Clinical Syndromes

Reference was made to FIG. 2B again. As shown in FIG. 2B, at 4 to 14 days after CSFV challenge, the difference of the clinical scores between the vaccinated and non-vaccinated pigs was statistically significant (P<0.05).

### (3) Weight Gain

Reference was made to FIG. 20, which was a bar graph showing average weight gain of pigs after vaccination with CSFV/PCV2 bivalent subunit vaccine according to an embodiment of the present invention. As shown in FIG. 2C, average weight gain (kg) of vaccinated (group A) pigs after virus challenge and before scarification was 9.1±1.0 kg, which was significantly higher than the one of the weight gain (group B, −1.0±0.55 kg). There was statistically significant difference between vaccinated and non-vaccinated pigs (P<0.05).

### (4) Virology

### (4.1) Serological ELISA for Detection of Antibody Titer Against CSFV

Reference was made to FIGS. 3A and 3B, which were line graphs respectively showing anti-CSFV antibody titers after CSFV challenge (FIG. 3A) or after PCV2 challenge (FIG. 3B) of CSFV/PCV2 bivalent subunit vaccine vaccinated pigs according to an embodiment of the present invention.

As shown in FIGS. 3A and 3B, all pigs had no anti-CSFV antibody [blocking percentage (Blocking %)≤30)] detected by ELISA before vaccination. After 2 weeks of vaccination of bivalent subunit vaccine, average anti-CSFV antibody titer (Blocking %) detected by ELISA in the sera of vaccinated (groups A and C) pigs increased to 62.7±2.5%, and antibody-positive (Blocking %≥40) pigs were 100%. After 4 weeks (before virus challenge) of vaccination of bivalent subunit vaccine, average anti-CSFV antibody titer of vaccinated (groups A and C) further increased to 83.1±1.7%, and antibody-positive pigs were 100% (10/10). Average anti-CSFV antibody titer (Blocking %) detected by ELISA in the sera of non-vaccinated (groups B and D) pigs was negative (0%; 0/10). There was statistically significant difference in the average anti-CSFV antibody titer detected by ELISA between vaccinated (groups A and C) and non-vaccinated (groups B and D) pigs at 2 and 4 weeks after vaccination (P<0.05), as shown in FIG. 3A.

After CSFV challenge, average anti-CSFV antibody titer in the sera of vaccinated (group A) and non-vaccinated (group B) pigs detected by ELISA showed continuously increase. Average anti-CSFV antibody titers in the sera of vaccinated (group A) pigs at the 4^{th}, 7^{th}, 10^{th} and 14^{th} day after virus challenge were 86.2±2.5%, 93.0±0.6%, 92.8±1.1% and 94.6±0.6%, respectively, all of which was higher than the average anti-CSFV antibody titers (3.0±1.6%, 13.0±3.1%, 20.3±7.3% and 27.2±5.7% at the 4^{th}, 7^{th}, 10^{th} and 14^{th} day after virus challenge) in the sera of non-vaccinated (group B) pigs. There was statistically significant difference between vaccinated and non-vaccinated pigs (P<0.05). Antibody-positive percentage of vaccinated (group A) pigs after virus challenge was 100% (5/5), and only one non-vaccinated (group B) pig was antibody-positive (20%, 1/5) at the 10^{th} and 14^{th} day after virus challenge, as shown in FIG. 3A.

At 2 to 7 weeks after PCV2 challenge, average anti-CSFV antibody titer in the sera of vaccinated (group C) pigs detected by ELISA maintained in the range of 87.6±1.3 to 88.5±1.0, and antibody-positive percentage of vaccinated (group C) pigs after virus challenge was 100% (5/5); however, average anti-CSFV antibody titer in the sera of non-vaccinated (group D) pigs detected by ELISA maintained in the range of 0.0±0.0 to 10.2±3.3, and all non-vaccinated pigs was antibody-negative. There was statistically significant difference in the average anti-CSFV antibody titer detected by ELISA between vaccinated (group C) and non-vaccinated (group D) pigs at 2 to 7 weeks after virus challenge (P<0.05), as shown in FIG. 3B.

### (4.2) CSFV Neutralizing Antibody Titer in the Serum

Reference was made to FIGS. 3C to 3D, which were line graphs respectively showing CSFV neutralizing antibody titer after CSFV challenge (FIG. 3C) or after PCV2 challenge (FIG. 3D) of CSFV/PCV2 bivalent subunit vaccine vaccinated pigs according to an embodiment of the present invention.

There was statistically significant difference between vaccinated and non-vaccinated pigs (P<0.05). Antibody-positive percentage of vaccinated (group A) after virus challenge were 100% (5/5), one non-vaccinated (group B) pig was antibody-positive (20%, 1/5) at the 10^{th} and 14^{th} day after virus challenge, as shown in FIG. 3A.

As shown in FIGS. 3C to 3D, all pigs (groups A and B in FIG. 3C; groups C and D in FIG. 3D) had no CSFV neutralizing antibody in the sera before vaccination. After 2 weeks of vaccination of bivalent subunit vaccine, average CSFV neutralizing antibody titer (Log₂) in the sera of vaccinated (groups A and C) increased to 0.9±0.5, and antibody-positive pigs were 30% (3/10). After 4 weeks (before virus challenge) of vaccination of bivalent subunit vaccine, average CSFV neutralizing antibody titer (Log₂) of vaccinated (groups A and C) pigs further increased to 5.4±0.3, and antibody-positive pigs were 100% (10/10). Average CSFV neutralizing antibody in the sera of non-vaccinated (groups B and D) pigs was negative. There was statistically significant difference in the average CSFV neutralizing antibody titers between vaccinated (groups A and C) and non-vaccinated (groups B and D) pigs at 4 weeks after vaccination (P<0.05).

After CSFV challenge, average CSFV neutralizing antibody titer (Log₂) in the sera of vaccinated (group A) pigs showed continuously increase. Average CSFV neutralizing antibody titers (Log₂) in the sera of vaccinated (group A) pigs at the 4^{th}, 7^{th}, 10^{th} and 14^{th} day after virus challenge were 6.9±0.6, 9.4±0.3, 12.1±0.5 and 11.3±0.3, respectively, and antibody-positive pigs were 100% (5/5). Average CSFV neutralizing antibody in the sera of non-vaccinated (group B) pigs was negative at 4 to 14 days after virus challenge. There was statistically significant difference in the average CSFV neutralizing antibody titers between vaccinated (groups A and C) and non-vaccinated (groups B and D) pigs at 4 to 14 days after virus challenge (P<0.05), as shown in FIG. 3C.

At 2 to 7 weeks after PCV2 challenge, average CSFV neutralizing antibody titers (Log₂) in the sera of vaccinated (group C) pigs increased to 5.7±0.5 to 6.5±0.3, and antibody-positive percentage of vaccinated (group C) pigs after virus challenge was 100%; however, CSFV neutralizing antibody in the sera of non-vaccinated (group D) pigs was negative. There was statistically significant difference in the average CSFV neutralizing antibody titers between vaccinated (group C) and non-vaccinated (group D) pigs at 2 to 7 weeks after virus challenge (P<0.05), as shown in FIG. 3D.

### (4.3) Serological ELISA for Detection of Antibody Titer

Reference was made to FIGS. 3E and 3F, which were line graphs respectively showing anti-PCV2 antibody titer after CSFV challenge (FIG. 3E) and anti-PCV2 antibody titer after PCV2 challenge (FIG. 3E) of CSFV/PCV2 bivalent subunit vaccine vaccinated pigs according to an embodiment of the present invention.

As shown in FIGS. 3E and 3F, all pigs (groups A, B, C and D) had no anti-PCV2 antibody [blocking percentage (S/P ratio<0.499) detected by ELISA before vaccination. After 4 weeks of vaccination of bivalent subunit vaccine, antibody-positive (S/P ratio≥0.500) vaccinated (groups A and C) pigs were 100%, and average anti-PCV2 antibody titer (S/P ratio) detected by ELISA in the sera of vaccinated (groups A and C) pigs was 1.2±0.1. There was statistically significant difference in the average anti-PCV2 antibody titer detected by ELISA between vaccinated (groups A and C) and non-vaccinated (groups B and D) pigs at 4 weeks after vaccination (P<0.05).

After PCV2 challenge, average anti-PCV2 antibody titer in the sera of vaccinated (group C) and non-vaccinated (group D) pigs detected by ELISA showed continuously increase. Average anti-PCV2 antibody titers in the sera of vaccinated (group C) pigs maintained in the range of 1.9±0.0 to 2.2±0.0, and antibody-positive vaccinated pigs were 100% (5/5). Average anti-PCV2 antibody titers in the sera of non-vaccinated (group D) pigs maintained in the range of 0.9±0.1 to 2.0±0.0, and antibody-positive vaccinated pigs were also 100% (5/5). The average anti-PCV2 antibody titers in the sera of vaccinated (group C) pigs were higher than the average anti-PCV2 antibody titers in the sera of non-vaccinated (group B) pigs at 2 to 6 weeks after virus challenge (P<0.05), as shown in FIG. 3E.

After CSFV challenge, average anti-PCV2 antibody titer in the sera of vaccinated (group A) pigs maintained in the range of 1.4±0.1 to 1.5±0.2 at the 4^{th}, 7^{th}, 10^{th} and 14^{th} day after virus challenge, and antibody-positive vaccinated pigs were also 100% (5/5). Average anti-PCV2 antibody in the sera of non-vaccinated (group B) pigs was negative (0%; 0/5). There was statistically significant difference in the average anti-PCV2 antibody titers between vaccinated (group A) and non-vaccinated (group B) pigs at 4 to 14 days after virus challenge (P<0.05), as shown in FIG. 3F.

### (4.4) Viral Nucleic Acid Titer in the Serum

Reference was made to FIGS. 4A to 4B, which were line graphs of CSF virus load (FIG. 4A) in the serum after CSFV challenge and PCV2 virus load (FIG. 4A) in the serum after PCV2 challenge of CSFV/PCV2 bivalent subunit vaccine vaccinated pigs according to an embodiment of the present invention.

As shown in FIGS. 4A to 4B, all pigs (groups A and B in FIG. 4A; groups C and D in FIG. 4B) had no CSFV nucleic acid or PCV2 nucleic acid in the sera before vaccination or virus challenge.

At 4 to 14 days after CSFV challenge, there was no nucleic acid detected in the sera of vaccinated (group A) and non-vaccinated (group B) pigs. Among pigs challenged with CSFV, viral nucleic acid in the serum was only found in one of vaccinated (group A) pigs at 4^{th} day after CSFV challenge and its titer was detected as 2.5 (Log₁₀ TCID₅₀/mL); however, CSFV viral nucleic acid titers in the serum of non-vaccinated (group B) pigs were detected as 4.6±0.3, 7.2±0.2, 8.1±0.2 and 7.8±0.1, at 4, 7, 10 and 14 day after CSFV challenge, respectively. There was statistically significant difference in the viral nucleic acid titers in the serum between vaccinated (group A) and non-vaccinated (group B) pigs (P<0.05), as shown in FIG. 4A.

The blood samples of pigs at 2, 3, 4 and 5 weeks after PCV2 challenge, and average viral nucleic acid concentrations (0.0±0.0, 0.0±0.0, 0.9±0.9, 0.0±0.0 Log₁₀ copies/mL) in the serum of vaccinated (group C) pigs were significantly less than the ones (5.2±0.2, 6.0±0.2, 5.6±0.4 and 5.7±0.2 Log₁₀ copies/mL) of non-vaccinated (group D) pigs. Among vaccinated (group C) and non-vaccinated (group D) pigs challenged with PCV2 at 2 to 7 weeks after PCV2 challenge, viral nucleic acid in the serum was not detected, as shown in FIG. 4B. At 6 and 7 weeks after PCV2 challenge, there was no statistically significant difference in the viral nucleic acid concentrations between vaccinated (group C) and non-vaccinated (group D) pigs (P>0.05), and the viral nucleic acid was negatively detected in the serum of all vaccinated (group C) pigs; however, it was viral nucleic acid-positive in few non-vaccinated (group D) pigs, two (40%, average viral nucleic acid concentration was decreased to 1.8±1.1 Log₁₀ copies/mL) or one (20%, average viral nucleic acid concentration was decreased to 0.9±0.9 Log₁₀ copies/mL) non-vaccinated pigs, as shown in FIG. 4B.

### (4.5) Virus Concentration in Tissues and Lymph Nodes

The viral nucleic acid concentrations (Log₁₀ TCID₅₀/mL) in the brain, tonsil, lung, spleen, hilar lymph node, mesenteric lymph node, groin lymph node and ileocecal valve of vaccinated (group A) pigs challenged with CSFV were 0.0±0.0, 4.7±0.5, 0.0±0.0, 1.2±0.7, 3.0±0.8, 3.1±0.8, 2.5±0.7 and 3.1±0.8, all of which was less than the ones (Log₁₀ TCID₅₀/mL) 6.5±0.3, 7.2±0.1, 7.0±0.2, 7.3±0.3, 7.2±0.2, 7.3±0.2, 7.3±0.2 and 7.3±0.2 of non-vaccinated (group B) pigs. There was statistically significant difference in the viral nucleic acid concentrations between vaccinated (group A) and non-vaccinated (group B) pigs (P<0.05). No PCV2 nucleic acid was detected in the organs of the vaccinated (group A) and non-vaccinated (group B) pigs challenged with CSFV.

The viral nucleic acid concentrations (Log₁₀ copies/g tissue) in the brain, tonsil, lung, spleen, hilar lymph node, mesenteric lymph node, groin lymph node and ileocecal valve of vaccinated (group C) pigs challenged with PCV2 were 0.7±0.3, 6.3±0.2, 7.4±0.4, 5.6±0.5 and 6.3±0.4, all of which was less than the ones (Log₁₀ copies/g tissue) 8.6±0.1, 9.4±0.2, 9.1±0.4, 8.8±0.3 and 9.1±0.3 of non-vaccinated (group D) pigs. There was statistically significant difference in the viral nucleic acid concentrations between vaccinated (group C) and non-vaccinated (group D) pigs (P<0.05). No CSFV nucleic acid was detected in the organs of the vaccinated (group C) and non-vaccinated (group D) pigs challenged with PCV2.

### (5) Biosafety of Vaccines

There were no apparent gross lesions and clinical syndromes of all vaccinated pigs.

(6) Effectiveness Test of CSFV/PCV2 Bivalent Vaccine-Vaccinated Pigs Challenged with PCV2
(6.1) Immunological Effectiveness of CSFV Challenge after Vaccination

### (6.1.1) Change in Body Temperature and Clinical Syndromes

Within 14 days after CSFV challenge, vaccinated (group A) pigs had normal body temperature, no characteristic clinical syndromes and apparent tissues lesions. Non-vaccinated (group B) pigs showed high body temperature, depression, chills and nervous symptoms.

### (6.1.2) Weight Gain

Average weight gain (kg) of vaccinated (group A) and non-vaccinated (group B) pigs from virus challenge to scarification were 9.1 kg and −1.0 kg, respectively, and the difference of the means between the two groups was statistically significant.

### (6.1.3) Antibody Titer in Sera

After 4 weeks of vaccination, average CSFV neutralizing antibody in the sera of vaccinated pigs were 100%, and the average antibody titer (Log₂) in the sera of vaccinated reached to 5.4. Average antibody titer of vaccinated pigs increased to 6.9 or more after CFSV virus challenge, and average antibody titer of vaccinated pigs maintained at 5.7 or more at 2 to 7 weeks after PCV2 challenge. Detection of antibodies using CSFV ELISA (Blocking %), antibody-positive pigs were 100% at 2 weeks after vaccination; the average antibody titer (Log₂) in the sera of vaccinated was 62.7 at 2 weeks after vaccination, and the average antibody titer (Log₂) in the sera of vaccinated was 83.1 at 4 weeks after vaccination. There was only one antibody-positive non-vaccinated pig after CSFV challenge.

### (6.1.4) Antibody Titer in Sera and Tissues

After CSFV challenge, there was no CSFV nucleic acid detected in the sera of vaccinated pigs; however, there were high CSFV nucleic acid titers, 4.6-8.1 Log₁₀ TCID₅₀/mL, detected in the non-vaccinated pigs. CSFV nucleic acid titers in brain, lung and lymph tissues of vaccinated pigs were at least 2.5-6.5 Log₁₀ TCID₅₀/mL less than the ones of non-vaccinated pigs.

(6.2) Immunological Effectiveness of PCV2 Challenge after Vaccination

### (6.2.1) Change in Body Temperature and Clinical Syndromes

Before PCV challenge, average body temperature of vaccinated (group C) and non-vaccinated (group D) pigs were 38.9±0.1° C. and 39.6±0.1° C., respectively. After PCV challenge, average body temperature of vaccinated (group C) and non-vaccinated (group D) pigs maintained below 40.5° C., vaccinated pigs had no apparently characteristic clinical syndromes. Non-vaccinated pigs revealed slightly fluffy hairs and average body temperature below 40.5° C.

### (6.2.2) Weight Gain

Average weight gain (kg) of vaccinated (group C) and non-vaccinated (group D) pigs from virus challenge to scarification were 31.2±1.8 kg and 26.8±2.3 kg, respectively, and the difference of the means between the two groups was not statistically significant (P>0.05).

### (6.2.3) Antibody Titer in Sera

Before PCV challenge, there was PCV2 neutralizing antibody-negative in the sera of non-vaccinated (group D) pigs. After 4 weeks of vaccination, average PCV2 neutralizing antibody in the sera of vaccinated pigs were 100%, and the average antibody titer (S/P ratio) in the sera of vaccinated pigs was 1.2. After PCV2 challenge, antibody detected by ELISA in the sera of vaccinated and non-vaccinated pigs continuously increased, and vaccinated and non-vaccinated pigs were antibody-positive (100%). At 2 to 4 weeks after PCV2 challenge, average antibody titer of vaccinated (group C) pigs were more than the one of non-vaccinated (group D) pigs, and the difference of the means between the two groups was statistically significant (P<0.05).

### (6.2.4) Antibody Titer in Sera and Tissues

Viral nucleic acid concentrations detected in the sera of vaccinated (group C) pigs were significantly less than the ones of non-vaccinated (group D) pigs from four blood collections at 2, 3, 4 and 5 weeks after PCV2 challenge. At 6 and 7 weeks after virus challenge, vaccinated (group C) pigs were viral nucleic acid-negative, and there was very low concentration of viral nucleic acid in the non-vaccinated (group D) pigs, and the difference of the means between the two groups was not statistically significant (P>0.05). Viral nucleic acid concentrations in lung, spleen and lymph nodes of vaccinated (group C) pigs were less than the ones of non-vaccinated (group D) pigs, and the difference of the means between the two groups was statistically significant.

The aforementioned examples demonstrated that CSFV/PCV2 bivalent vaccine could be safely administered to 5-weeks-old piglets via a single vaccination and confer effectively immune protection without boost vaccination, thereby significantly increasing neutralizing antibody titers and ELISA-based antibody titers against CSFV and PCV2 in the vaccinated pigs, as well as effectively inhibiting the viral proliferation in the body and clinical syndromes.

In summary, the aforementioned specific amino acid sequences, specific processes, specific compositions, specific analysis models or specific evaluation methods were only exemplary to describe the porcine bivalent subunit vaccine composition in a single dose against CSFV and PCV2. However, those of common knowledge in the technical field of the present invention should understand that other amino acid sequences, other processes, other compositions, other analysis models or other evaluation methods, etc., also can be applied to the porcine bivalent subunit vaccine composition in a single dose against CSFV and PCV2, without limiting to the aforementioned description of the present invention.

For example, the soluble bivalent antigens consisting of CSFV-E2 recombinant protein and PCV2-ORF2 recombinant protein can be expressed by other sequences, the vaccine composition can be produced by CpG adjuvant of other sequence and/or other dual phase adjuvants, for optimizing the process and the mass production. In addition to the porcine bivalent subunit vaccine composition in a single dose, the resultant bivalent antigens can also be applied to rapid test kits, biochips or other commercially available examination products, depending on the actual requirements.

According to the aforementioned embodiments, the porcine bivalent subunit vaccine composition in a single dose can include soluble bivalent antigens beneficially expressed by mammalian cell expression system, CpG adjuvant and a dual phase adjuvant. The porcine bivalent subunit vaccine composition in a single dose can be administered via a single vaccination and confer effectively immune protection without boost vaccination, thereby significantly eliminating the possibility of simultaneous occurrence of CSFV and PCV in the field, reducing the failure of the current LPC vaccine in controlling CSFV, decreasing the risk of transmission of wild CSFV to pig farms, and elevating the opportunity of removing CSFV.

Although the present invention has been described in considerable detail with reference to certain embodiments thereof, other embodiments are possible. Therefore, the spirit and scope of the appended claims should not be limited to the description of the embodiments contained herein.

What is claimed is:

1. A porcine bivalent subunit vaccine composition in a single dose, comprising:
a bivalent antigen consisted of E2 recombinant protein and ORF2 recombinant protein, an amino acid sequence of the E2 recombinant protein is listed as SEQ ID NO:1, and the ORF2 recombinant protein is encoded by a nucleic acid sequence listed as SEQ ID NO:2;
a CpG adjuvant consisted of a nucleic acid sequence listed as SEQ ID NO:3; and
a dual phase adjuvant, and
wherein the porcine bivalent subunit vaccine composition is formulated for a single-dose administration to a subject in need thereof.

2. The porcine bivalent subunit vaccine composition in the single dose of claim 1, wherein the E2 recombinant protein is derived from classical swine fever virus (CSFV) subgroup 2.1a.

3. The porcine bivalent subunit vaccine composition in the single dose of claim 1, wherein the ORF2 recombinant protein is derived from porcine circovirus type 2 (PCV2).

4. The porcine bivalent subunit vaccine composition in the single dose of claim 1, wherein the bivalent antigen is produced by a mammalian cell expression system.

5. The porcine bivalent subunit vaccine composition in the single dose of claim 1, wherein the E2 recombinant protein is encoded by a nucleic acid sequence listed as SEQ ID NO:4.

6. The porcine bivalent subunit vaccine composition in the single dose of claim 1, wherein an effective dose of the E2 recombinant protein is 25 μg/mL to 50 μg/mL.

7. The porcine bivalent subunit vaccine composition in the single dose of claim 6, wherein an effective dose of the ORF2 recombinant protein is 25 μg/mL to 75 μg/mL.

8. The porcine bivalent subunit vaccine composition in the single dose of claim 1, wherein a weight ratio of the CpG adjuvant and the bivalent antigen is 1:1 to 1:2.

9. The porcine bivalent subunit vaccine composition in the single dose of claim 1, wherein the dual phase adjuvant is a water-in-oil-in-water (W/O/W) adjuvant.

10. The porcine bivalent subunit vaccine composition in the single dose of claim 1, wherein an effective dose of the dual phase adjuvant is 50 vol. % based on the porcine bivalent subunit vaccine composition.

11. A porcine bivalent subunit vaccine composition in a single dose, comprising:
a bivalent antigen consisted of E2 recombinant protein and ORF2 recombinant protein, an amino acid sequence of the E2 recombinant protein is listed as SEQ ID NO:1, and the ORF2 recombinant protein is encoded by a nucleic acid sequence listed as SEQ ID NO:2, an effective dose of the E2 recombinant protein is 25 μg/mL to 50 μg/mL and an effective dose of the ORF2 recombinant protein is 25 μg/mL to 75 μg/mL;
a CpG adjuvant consisted of a nucleic acid sequence listed as SEQ ID NO:3; and
a dual phase adjuvant, and
wherein the porcine bivalent subunit vaccine composition is formulated for a single-dose administration to a subject in need thereof.

12. The porcine bivalent subunit vaccine composition in the single dose of claim 11, wherein the bivalent antigen is produced by a mammalian cell expression system.

13. The porcine bivalent subunit vaccine composition in the single dose of claim 11, wherein an effective dose of the E2 recombinant protein is 25 μg/mL.

14. The porcine bivalent subunit vaccine composition in the single dose of claim 11, wherein an effective dose of the ORF2 recombinant protein is 50 μg/mL.

15. The porcine bivalent subunit vaccine composition in the single dose of claim 1, wherein a weight ratio of the CpG adjuvant and the bivalent antigen is 1:1 to 1:2.

16. A porcine bivalent subunit vaccine composition in a single dose, comprising:
a bivalent antigen consisted of E2 recombinant protein and ORF2 recombinant protein, an amino acid sequence of the E2 recombinant protein is listed as SEQ ID NO:1, and the ORF2 recombinant protein is encoded by a nucleic acid sequence listed as SEQ ID NO:2, an effective dose of the E2 recombinant protein is 25 μg/mL and an effective dose of the ORF2 recombinant protein is 50 μg/mL;
a CpG adjuvant consisted of a nucleic acid sequence listed as SEQ ID NO:3; and
a dual phase adjuvant, and
wherein the porcine bivalent subunit vaccine composition is formulated for a single-dose administration to a piglet without infections of CSFV and/or PCV.

17. The porcine bivalent subunit vaccine composition in the single dose of claim 11, wherein the bivalent antigen is produced by a mammalian cell expression system.

18. The porcine bivalent subunit vaccine composition in the single dose of claim 1, wherein a weight ratio of the CpG adjuvant and the bivalent antigen is 1:1 to 1:2.

19. The porcine bivalent subunit vaccine composition in the single dose of claim 16, wherein the dual phase adjuvant is a water-in-oil-in-water (W/O/W) adjuvant.

20. The porcine bivalent subunit vaccine composition in the single dose of claim 16, wherein an effective dose of the dual phase adjuvant is 50 vol. % based on the porcine bivalent subunit vaccine composition.
